(19) United States
(12) Patent Application Publication
(10) Pub. No.: US 2009/0215704 A1
(43) Pub. Date: Aug. 27, 2009

(54) HEALTH-RELATED LIQUID COMPOSITIONS

(76) Inventors: Janice M. Johnson, Eden Prairie, MN (US); John Q Hunsicker, Minneapolis, MN (US); Karla J. Nelson, Minneapolis, MN (US); Laura H. Crosby, Eden Prairie, MN (US)

Correspondence Address: CARGILL, INCORPORATED, P.O. Box 5624, MINNEAPOLIS, MN, 55440-5624, US

(21) Appl. No.: 11/908,905

(22) PCT Filed: Mar. 6, 2006
(86) PCT No.: PCT/US06/07921

§ 371 Date: Oct. 10, 2008

Related U.S. Application Data
(60) Provisional application No. 60/662,705, filed on Mar. 17, 2005.

(51) Int. Cl. A61K 31/7048; A61K 31/715; A61K 31/702; A61P 3/10

(52) U.S. Cl. 514/25; 514/54; 514/62

(57) ABSTRACT

There are disclosed liquid compositions comprising water, a fiber such as inulin, and a glycosaminoglycan that is selected from chondroitin sulfate, glucosamine, or a mixture thereof. The liquid compositions may further comprise at least one or more sweeteners, a source of calcium, at least one or more flavor additives, trehalose, at least one or more acids, sodium citrate, monopotassium phosphate, fruit juice, fruit juice concentrate, or the like, and any combination thereof. The liquid compositions may be suitable for use in health-related applications, among other applications.

### FIELD OF DISCLOSURE

The present disclosure relates to health-related liquid compositions that are expected to provide, for example, joint health benefits.

### BACKGROUND

Health-related liquid compositions are known. However, there is a continuing need to provide health-related liquid compositions that offer other benefits.

### SUMMARY OF THE DISCLOSURE

This disclosure relates to liquid compositions comprising water, a soluble fiber, such as preferably inulin, and chondroitin sulfate or glucosamine, or both. Other ingredients such as a source of calcium, a flavor, a sweetener, citric acid, malic acid, sodium citrate dihydrate, monopotassium phosphate, trehalose, fruit juice, fruit juice concentrate, and the like, may be incorporated into the liquid compositions.

### DETAILED DESCRIPTION OF THE DISCLOSURE

This disclosure relates to liquid compositions comprising water, a soluble fiber such as preferably inulin and chondroitin sulfate or glucosamine or both. Other ingredients such as a source of calcium, a flavor, a sweetener, citric acid, malic acid, sodium citrate dihydrate, monopotassium phosphate, trehalose, fruit juice, fruit juice concentrate, and the like, may be incorporated into the liquid compositions.

In one embodiment, the disclosure relates to a liquid composition comprising water, a soluble fiber such as inulin, and chondroitin sulfate. Other ingredients such as sweeteners, sources of calcium such as calcium lactate, trehalose, flavors, citric acid, malic acid, sodium citrate, monopotassium phosphate, fruit juice, fruit juice concentrate, and the like, may be incorporated into the liquid composition.

In another embodiment, the disclosure relates to a liquid composition comprising water, a soluble fiber such as inulin, and glucosamine. Other ingredients such as sweeteners, sources of calcium such as calcium lactate, trehalose, flavors, citric acid, malic acid, sodium citrate, monopotassium phosphate, fruit juice, fruit juice concentrate, and the like, may be incorporated into the liquid compositions.

In another embodiment, the disclosure relates to a liquid composition comprising water, a soluble fiber such as inulin, chondroitin sulfate, and glucosamine. Other ingredients such as sweeteners, sources of calcium such as calcium lactate, trehalose, flavors, citric acid, malic acid, sodium citrate, monopotassium phosphate, fruit juice, fruit juice concentrate, and the like, may be incorporated into the liquid composition.

In another embodiment, the disclosure relates to a liquid composition comprising about 94.7% water, about 2.0% erythritol, about 1.4% inulin, about 0.78% calcium lactate, about 0.5% trehalose, about 0.08% natural lemon, about 0.08% natural lime, about 0.05% citric acid, about 0.05% malic acid, about 0.04% sodium citrate, dihydrate, about 0.02% acesulfame potassium artificial sweetener, about 1% solution, about 0.01% monopotassium phosphate, and about 0.17% chondroitin sulfate.

In another embodiment, the disclosure relates to a liquid composition comprising about 94.7% water, about 2.0% erythritol, about 1.4% inulin, about 0.78% calcium lactate, about 0.50% trehalose, about 0.13% peach flavor, about 0.05% citric acid, about 0.05% malic acid, about 0.04% sodium citrate, dihydrate about 0.02% acesulfame potassium artificial sweetener, 1% solution, about 0.01% monopotassium phosphate, and about 0.17% chondroitin sulfate.

In the embodiments of the disclosure, the following is applicable.

Any source of soluble fiber may be utilized in producing the liquid compositions. Conventional sources of soluble fibers are gums, hydrocolloids, mucilages, hemicellulose, pectins, and the like, and mixtures thereof. Suitable sources of soluble fiber include, but are not limited to, gum arabic, locust bean gum, polydextrose, inulin, arabinogalactose, hydrolyzed guar gum, xanthan, pectin, alginate, carrageenan, beta-glucans, tragacanth, arabinoxylan, or the like, or mixtures thereof.

In producing the liquid compositions of the present disclosure, there are preferred amounts of fiber, chondroitin sulfate, glucosamine, and calcium, when used. More particularly, it is preferred to use fiber in an amount ranging from greater than 0% to about 10% (w/w); chondroitin sulfate in an amount ranging from greater than 0% to about 0.5% (w/w); glucosamine in an amount ranger from greater than 0% to about 0.7% (w/w); and calcium in an amount ranging from greater than 0% to about 0.5% (w/w).

Any source of calcium is suitable for use in the liquid compositions of the disclosure. Examples of suitable sources of calcium include, but are not limited to, calcium lactate, calcium gluconate, a combination of calcium lactate and calcium gluconate, tricalcium phosphate, dicalcium phosphate, and the like, and mixtures thereof.

Suitable for use as buffers in the liquid compositions of the present disclosure, but not limited thereto, are potassium citrate, sodium citrate, potassium phosphate, sodium phosphate, and the like, and mixtures thereof.

Any sweetener may be used in the liquid compositions of the present disclosure. Examples of suitable sweeteners include, but are not limited to, high intensity sweeteners such as sucralose, acesulfame potassium, aspartame, stevia, thaumatin, neotame, MONATIN brand high intensity sweetener and the like, or mixtures thereof, corn syrups, high fructose corn syrups, sugar alcohols such as maltitol, erythritol, lactitol, xylitol, sucrose, fructose, dextrose, and the like, and mixtures thereof.

Any acid may be used in the liquid compositions of the present disclosure. Included in the suitable acids, but not limited thereto, are phosphoric acid, malic acid, citric acid, lactic acid and the like, and mixtures thereof.

Any fruit juice or fruit juice concentrate may be used in the liquid compositions of the disclosure. Exemplary fruit juice and fruit juice concentrate include, but are limited to, apple, orange, banana, strawberry, cranberry, kiwi, blueberry, raspberry, grape, mango, grapefruit, or the like. The fruit juices or fruit juice concentrate may be used alone or in combination.

The buffer, sweetener, fruit juice, fruit juice concentrate, and acid are used in any amount conventionally utilized in producing health-related liquid compositions of the type previously available.

The liquid compositions of the present disclosure may be prepared by any conventional technique. In the present disclosure, the liquid compositions of the Examples were prepared by the following process:
(a) Water was metered, or weighed, into a mixing tank;
(b) The fiber, which was inulin or beta-glucan in the Examples, was added to the water which was being agitated;
(c) Thereafter, chondroitin sulfate, or glucosamine, or both, if used, was added to the water-inulin mixture;
(d) Thereafter the fruit juice or fruit juice concentrate, or both, was added to the mixture;
(e) Then calcium lactate was added to the resultant mixture;
(f) Then sodium citrate was added to the mixture;
(g) The monopotassium phosphate was added to the mixture;
(h) The sweeteners, which in the Examples included erythritol, trehalose, and acesulfame potassium, were added to the mixture;
(i) Then flavors, which in the Examples were lemon, lime, or peach, were added to the mixture;
(j) Then citric acid and malic acid were added to the mixture;
(k) The pH of the mixture was measured and adjusted, if necessary, to a target pH of 4.0±0.1; and
(l) Then the mixture was filtered to provide the liquid composition of the disclosure.

The following examples are presented to illustrate the present disclosure and to assist one of ordinary skill in making and using the same. The examples are not intended in any way to otherwise limit the scope of the disclosure.

### EXAMPLE 1

In this Example there is produced a lemon lime flavored health-related liquid composition, using the procedure set forth herein.

The liquid composition was prepared by mixing 94.7% (3585.0 g) water; 2.0% (75.7 g) erythritol; 1.4% (53 g) inulin, 0.78% (29.5 g) calcium lactate; 0.50% (18.9 g) trehalose; 0.08% (2.9 g) natural lemon flavor; 0.08% (2.9 g) natural lime flavor; 0.05% (1.9 g) citric acid; 0.05% (1.9 g) malic acid; 0.04% (1.5 g) sodium citrate, dihydrate; 0.02% (0.76 g) acesulfame potassium; 0.01% (0.4 g) monopotassium phosphate; and 0.17% (6.4 g) chondroitin sulfate. All percentages are weight/weight (w/w).

### EXAMPLE 2

In this Example, there is produced a peach flavored health-flavored liquid composition, using the procedure set forth herein:

The liquid composition was prepared by mixing 94.7% (3585.8 g) water; 2.0% (75.7 g) erythritol; 1.4% (53 g) inulin, 0.78% (29.5 g) calcium lactate; 0.50% (18.9 g) trehalose; 0.13% (5 g) peach flavor; 0.05% (1.9 g) citric acid; 0.05% (1.9 g) malic acid; 0.04% (1.5 g) sodium citrate, dihydrate; 0.02% (0.76 g) acesulfame potassium; 0.01% (0.4 g) monopotassium phosphate; and 0.17% (6.4 g) chondroitin sulfate. All percentages are weight/weight (w/w).

### EXAMPLE 3

In this Example, there is produced a health-related liquid composition comprising water, inulin, and chondroitin sulfate, using the procedure set forth herein.

The liquid composition was prepared by mixing 98.4% (4921.5 g) water; 1.4% (70 g) inulin; and 0.17% (8.5 g) chondroitin sulfate. All percentages are weight/weight (w/w)

### EXAMPLE 4

In this Example, there is produced a health-related liquid composition comprising water, inulin, and glucosamine, using the procedure set forth herein.

The liquid composition was prepared by mixing 98.4% (4920.7 g) water; 1.4% (70 g) inulin; and 0.19% (9.4 g) glucosamine. All percentages are weight/weight (w/w)

### EXAMPLE 5

In this Example, there is produced a health-related liquid composition comprising water, inulin, chondroitin sulfate, and glucosamine, using the procedure set forth herein.

The liquid composition was prepared by mixing 98.2% (4912.2 g) water; 1.4% (70 g) inulin; 0.17% (8.5 g) chondroitin sulfate; and 0.19% (9.4 g) glucosamine. All percentages are weight/weight (w/w)

### EXAMPLE 6

In this Example, there is produced a health-related liquid composition comprising water, inulin, chondroitin sulfate, glucosamine, and calcium lactate, using the procedure set forth herein.

The liquid composition was prepared by mixing 97.5% (4873.2 g) water; 1.4% (70 g) inulin; 0.17% (8.5 g) chondroitin sulfate; 0.19% (9.4 g) glucosamine; and 0.78% (39 g) calcium lactate. All percentages are weight/weight (w/w)

### EXAMPLE 7

In this Example, there is produced a health-related fruit juice-containing liquid composition comprising water, cranberry juice concentrate, white grape juice concentrate, inulin, chondroitin sulfate, glucosamine, and calcium lactate, using the procedure set forth herein.

The liquid composition was prepared by mixing 76.2% (3811.7 g) water; 4.1% (202.5 g) cranberry juice concentrate; 17.2% (859 g) white grape juice concentrate; 1.4% (70 g) inulin; 0.17% (8.5 g) chondroitin sulfate; 0.19% (9.4 g) glucosamine; and 0.78% (39 g) calcium lactate. All percentages are weight/weight (w/w)

### EXAMPLE 8

In this Example, there is produced a health-related fruit juice-containing liquid composition comprising orange juice, beta-glucan fiber, chondroitin sulfate, glucosamine, and calcium lactate, using the procedure set forth herein.

The liquid composition was prepared by mixing 98.3% (4916.4 g) orange juice, that includes water, 0.54% (26.7 g) beta-glucan, 0.17% (8.5 g) chondroitin sulfate; 0.19% (9.4 g) glucosamine; and 0.78% (39 g) calcium lactate. All percentages are weight/weight (w/w).

All of the liquid compositions of Examples 1 through 8 were evaluated for taste, and were found to be acceptable for consumption.

This disclosure has been described with reference to various specific and illustrative embodiments and techniques. However, one skilled in the art will recognize that many variations and modifications may be made while retaining within the spirit and scope of the disclosure and the claims.

We claim:

1. A liquid composition comprising water, a fiber, and a glycosaminoglycan selected from the group consisting of chondroitin sulfate, glucosamine, and mixtures thereof.

2. The liquid composition according to claim 1 wherein the fiber is present in an amount of greater than zero (0) to about 10% (weight/weight), the chondroitin sulfate is present in an amount of greater than zero (0) to about 0.5% weight/weight, and the glucosamine is present in an amount of greater than zero (0) to about 0.7% weight/weight.

3. The liquid composition according to claim 1 wherein the fiber is selected from the group consisting of inulin, polydextrose, arabinogalactose, hydrolyzed guar gum, gum arabic, locust bean gum, xanthan, pectin, alginate, carageenan, beta-glucans, tragacanth, arabinoxylan, and mixtures thereof.

4. The liquid composition according to claim 1 wherein the fiber is inulin.

5. The liquid composition according to claim 1 wherein the glycosaminoglycan is chondroitin sulfate.

6. The liquid composition according to claim 1 wherein the fiber is inulin and the glycosaminoglycan is chondroitin sulfate.

7. The liquid composition according to claim 1 wherein the glycosaminoglycan is glucosamine.

8. The liquid composition according to claim 1 wherein the fiber is inulin and the glycosaminoglycan is glucosamine.

9. The liquid composition according to claim 1 wherein the glycosaminoglycan is a mixture of chondroitin sulfate and glucosamine.

10. The liquid composition according to claim 1 wherein the fiber is inulin, and the glycosaminoglycan is a mixture of chondroitin sulfate and glucosamine.

11. The liquid composition according to claim 1 further comprising a source of calcium.

12. The liquid composition according to claim 11 wherein the source of calcium is present in an amount of greater than zero (0) to about 0.5% weight/weight.

13. The liquid composition according to claim 6 further comprising a source of calcium.

14. The liquid composition according to claim 8 further comprising a source of calcium.

15. The liquid composition according to claim 10 further comprising a source of calcium.

16. The liquid composition according to claim 1 further comprising a component selected from the group consisting of at least one sweetener, a source of calcium, at least one flavor additive, at least one acid, sodium citrate, trehalose, monopotassium phosphate, at least one fruit juice, at least one fruit juice concentrate, and mixtures thereof.

17. The liquid composition according to claim 16 wherein the sweetener comprises a mixture of erythritol and acesulfame potassium, the source of calcium comprises calcium lactate, and the acid comprises a mixture of citric acid and malic acid.

18. The liquid composition according to claim 17 wherein the fiber is inulin.

19. The liquid composition according to claim 18 wherein the glycosaminoglycan is chondroitin sulfate.

20. The liquid composition according to claim 18 wherein the glycosaminoglycan is glucosamine.

21. The liquid composition according to claim 18 wherein the glycosaminoglycan is a mixture of chondroitin sulfate and glucosamine.

22. The liquid composition according to claim 16 wherein the sweetener comprises a mixture of erythritol and high fructose corn syrup.
